(19) United States
(12) Patent Application Publication
(10) Pub. No.: US 2014/0107460 A1
(43) Pub. Date: Apr. 17, 2014

(54) Image-Guided Radiotherapy For Internal Tumor Boost

(71) Applicant: Nam Nguyen, Tucson, AZ (US)

(72) Inventor: Nam Nguyen, Tucson, AZ (US)

(21) Appl. No.: 13/652,821

(22) Filed: Oct. 16, 2012

(51) Int. Cl. A61B 6/00; A61N 5/10

(52) U.S. Cl. 600/407

(57) ABSTRACT

The present invention features an image-guided radiotherapy method for tumor treatment. The method comprises the steps of obtaining a three-dimensional visualized tumor image, identifying the boundary of the tumor; designating and applying a boosted radiation dose of treatment for a boost region within the tumor boundary, wherein a predetermined safety region is between the boosted region and the tumor boundary with a predetermined minimum distance between the boosted region boundary and tumor boundary.

### FIELD OF THE INVENTION

The present invention related to image-guided radiotherapy, and more particularly to image-guided radiotherapy for tumor treatment.

### BACKGROUND OF THE INVENTION

Radiotherapy is a proven modality for cancer cure similar to surgery for tumors of all sites. The probability to destroy the cancer locally is proportional to the radiation dose delivered to the cancer sites. Most often, the effectiveness of radiotherapy is limited by the radiation dose that can safely be delivered to the normal organs adjacent to the tumor. Serious complications may occur if the normal organs receive a radiation dose that exceeds their tolerance to radiation. Paralysis (spinal cord injury), blindness (optic nerve injury), stroke (brain injury), bleeding (blood vessels injury), inflammation of lungs (lungs injury) and bowels (bowels injury) may lead to death or seriously affect patient quality of life are well known complications of radiation treatment.

On Section 6.4.2.4 Radiation Therapy Oncology Group (RTOG) study number 0225: A Phase II Study of Intensity Modulated Radiation Therapy (IMRT)+/Chemotherapy for Nasopharyngeal Cancer, it is specified that “No more than 20% of any PTV₇₀ (the gross tumor volume with a 5 mm margin) will receive ≧110% of the prescribed dose.” The rule limits the toxicity of the treatment to avoid complication.

As used herein, the term “prescribed dose” means the conventional dose established in the literature for cancer cure with external beam radiotherapy alone or combined with chemotherapy for locally advanced head and neck cancer. As non-limiting examples, the “prescribed dose” for Oropharyngeal cancer, Oral cavity cancer, Laryngeal cancer, Hypopharyngeal cancer is about 7000 cGy, at about 200 cGy per day.

FIG. 1 shows a schematic view for a gross tumor volume (GTV), a clinical tumor volume (CTV) and planning target volume (PTV), wherein the planning target volume (PTV) is the traditional radiation treatment volume and is beyond tumor boundary. In FIG. 1, a tumor (110) has a tumor boundary (115) which encloses the gross tumor boundary. Clinical tumor volume is enclosed by GTV boundary (116) and planning tumor volume is enclosed by PTV boundary (118).

FIG. 2 shows schematic representation of “volumes” in radiation therapy in terms of Gross Target Volume, Clinical Target Volume, Planning Target Volume from Page 5, Chapter 1: The Discipline of Radiation Oncology, Book: Perez and Brady's Principles and Practice of Radiation Oncology, 5th Edition, published by Lippincott Williams & Wilkins with ISBN-10: 078176369X. This figure clearly shows that the planning target volume (PTV) is beyond tumor boundary.

FIG. 3 shows the Memorial Sloan-Kettering Cancer Center (MSKCC) Clinical Dose Limits and Inverse Planning Algorithm Constraints for Primary Nasopharynx Tumors, excerpted from book “A practical guide to intensity-modulated radiation therapy” (Medical Physics Pub., 2003, ISBN: 1930524137), Chapter 10: IMRT for head and neck Cancer, Table 10.3, page 201. The table clearly regulates that the maximum dose is 105%.

FIG. 4 shows the compliance criteria of radiation treatment in Radiation Therapy Oncology Group (RTOG) study number 0920: A Phase III Study of Postoperative Radiation Therapy (IMRT)+/−Cetuximab for Locally-Advanced Resected Head and Neck Cancer, section 6.7, page 27. The criteria lists in Row 1 that any Radiation dose (RT)>66 Gy as a major variation should be avoided at any rate. The 66 Gy corresponds to a 10% increase over PTV 60 Gy (PTV: planning target volume).

FIG. 5 shows the Critical Normal Structures in Radiation Therapy Oncology Group (RTOG) study number 0225: A Phase II Study of Intensity Modulated Radiation Therapy (IMRT)+/Chemotherapy for Nasopharyngeal Cancer, section 6.4.3 Critical Normal Structures, page 7. The Critical Normal Structures discloses clearly that 60 Gy or 1% of the PTV cannot exceed 65 Gy (which is close to 10% increase over PTV 60 Gy radiation.)

FIG. 6 shows the hotspot radiation regulation in a presentation (slide 13) of a research taken at Dana-Farber/Brigham & Women's Cancer Center and Harvard Medical School (“Variability in planning criteria and plan evaluation”, Laurence Court, the American Association of Physicists in Medicine annual meeting 2010). The slide clearly shows the aiming for hotspots radiation is limited to <110% of the radiation dose.

On the other hand, low radiation dose is ineffective for cancer cure and the patient dies from uncontrolled tumor growth or from complications resulting from tumor destruction of the normal organs. Thus, the clinician is often faced with a dilemma; either let the cancer kill the patient or exposes the patient to serious injury from radiation complications. Therefore, there is a need for a balanced method for image-guided radiotherapy for providing higher dose for tumor tissues and avoiding excessive radiation to normal tissues.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features an image-guided radiotherapy method for tumor treatment. The method comprises the steps of obtaining a three-dimensional visualized tumor image, identifying the boundary of the tumor; designating and applying a boosted radiation dose of treatment for a boost region within the tumor boundary, wherein a predetermined safety region is between the boosted region and the tumor boundary with a predetermined minimum distance between the boosted region boundary and tumor boundary; designating a predetermined prescribed radiation dose of treatment for the boundary of the tumor, wherein the predetermined prescribed radiation dose is smaller than the boosted radiation dose; applying radiation treatment for the region safety region with a first dose decreasing rate such that the radiation dose on the tumor boundary is the a predetermined prescribed radiation dose; applying radiation treatment beyond the tumor boundary and within the radiation boundary with a second dose decreasing rate.

### BRIEF DESCRIPTION OF THE DRAWINGS

FIG. 1 shows a schematic view of GTV, CTV and PTV.

FIG. 2 shows schematic representation of “volumes” in radiation therapy.

FIG. 3 shows the Memorial Sloan Kettering Cancer Center (MSKCC) Clinical Dose Limits and Inverse Planning Algorithm Constraints for Primary Nasopharynx Tumors.

FIG. 4 shows the compliance criteria of radiation treatment adopted in Radiation Therapy Oncology Group (RTOG) study number 0920.

FIG. 5 shows the Critical Normal Structures in Radiation Therapy Oncology Group (RTOG) study number 0225.

FIG. 6 shows the hotspot radiation regulation in a presentation (slide 13) of a research taken at Dana-Farber/Brigham & Women's Cancer Center and Harvard Medical School.

FIG. 7 shows a brief view of a tumor boundary and boosted treatment region of the present invention.

FIG. 8 shows a brief view of a tumor boundary and boosted treatment region when a radiation sensitive normal organ is near the tumor boundary of the present invention.

FIG. 9 shows a front view comparison of radiation treatment results of the present method before and after treatment.

FIG. 10 shows a side view comparison of radiation treatment results before and after treatment.

FIG. 11 shows a radiation dose comparison between a traditional method and the present method.

FIG. 12 shows some typical radiation dose for typical RTOG Guideline method and the present method for Radiation Sensitive Tumors.

FIG. 13 shows some typical radiation dose for typical RTOG Guideline method and the present method for Radiation Resistant Tumors.

### DESCRIPTION OF PREFERRED EMBODIMENTS

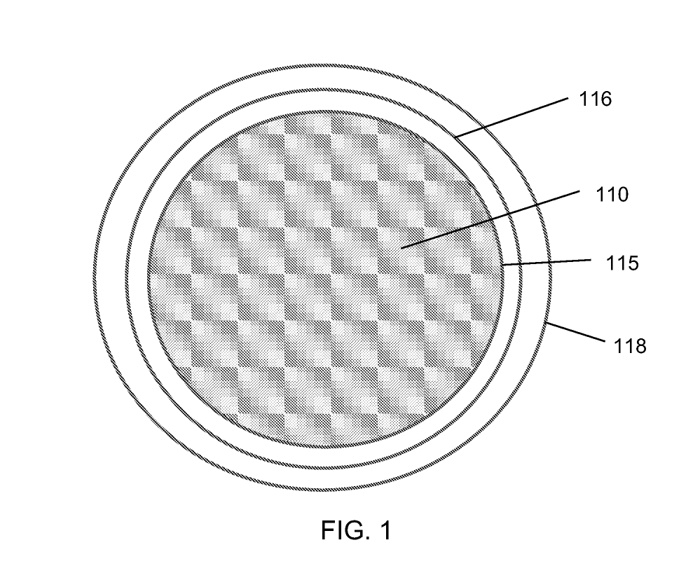

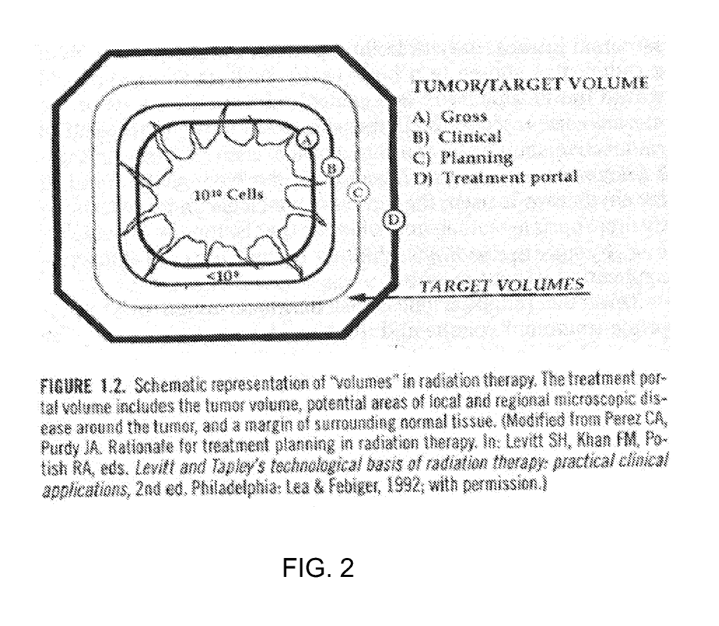

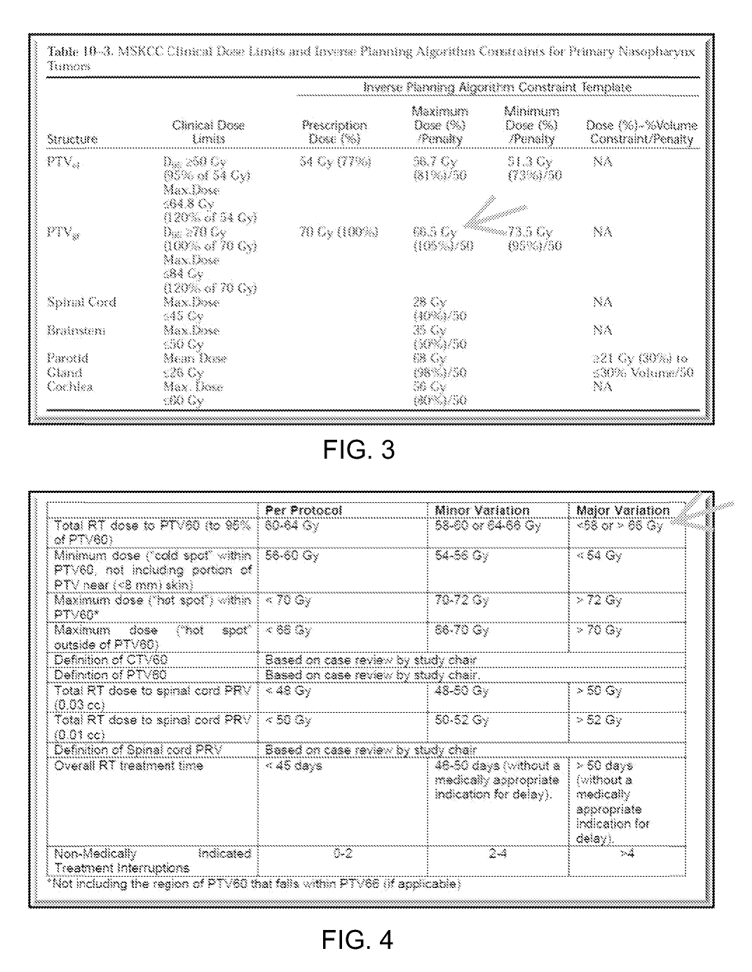

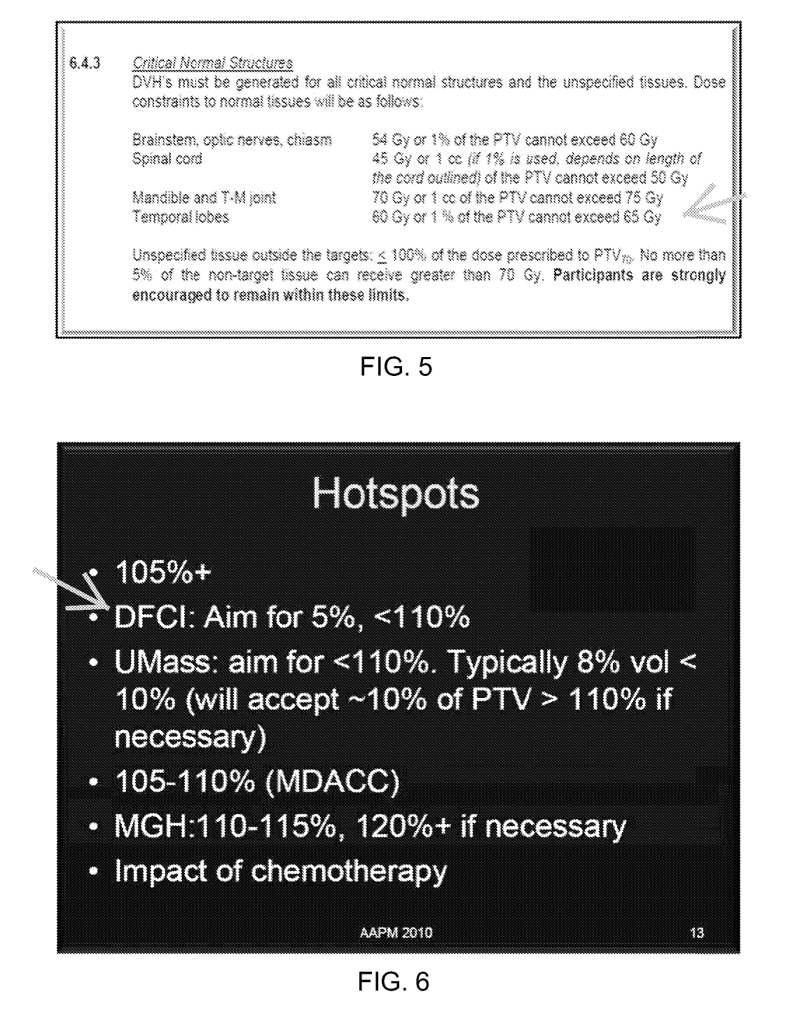

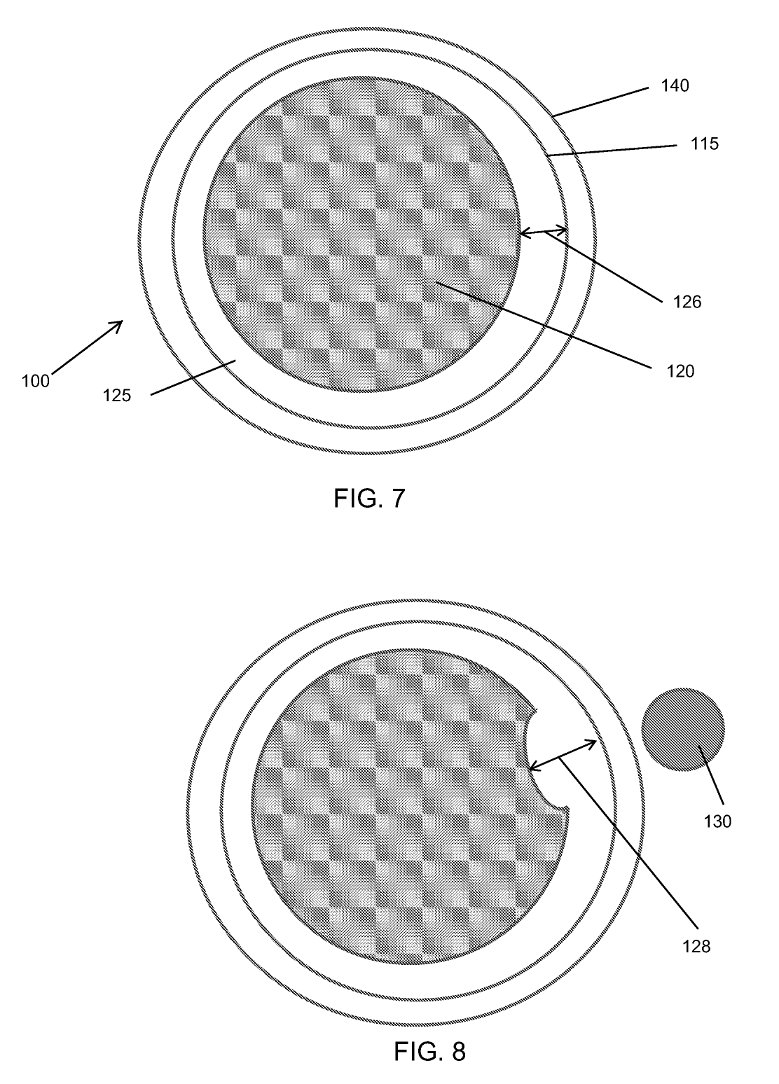

Referring now to FIG. 1-13, the present invention features an image-guided radiotherapy method for tumor treatment. The method comprises the steps of: a) obtaining a three-dimensional visualized tumor image (100); b) identifying the boundary (115) of the tumor (110); b) designating and applying a boosted radiation dose of treatment for a boost region (120) within the tumor boundary, wherein a predetermined safety region (125) is between the boosted region and the tumor boundary (115) with a predetermined minimum distance (126) between the boosted region boundary and tumor boundary, wherein the boosted region (120) is more than 20% of the volume within the tumor boundary (115). Steps (a) through (c) are repeated for new treatment each time.

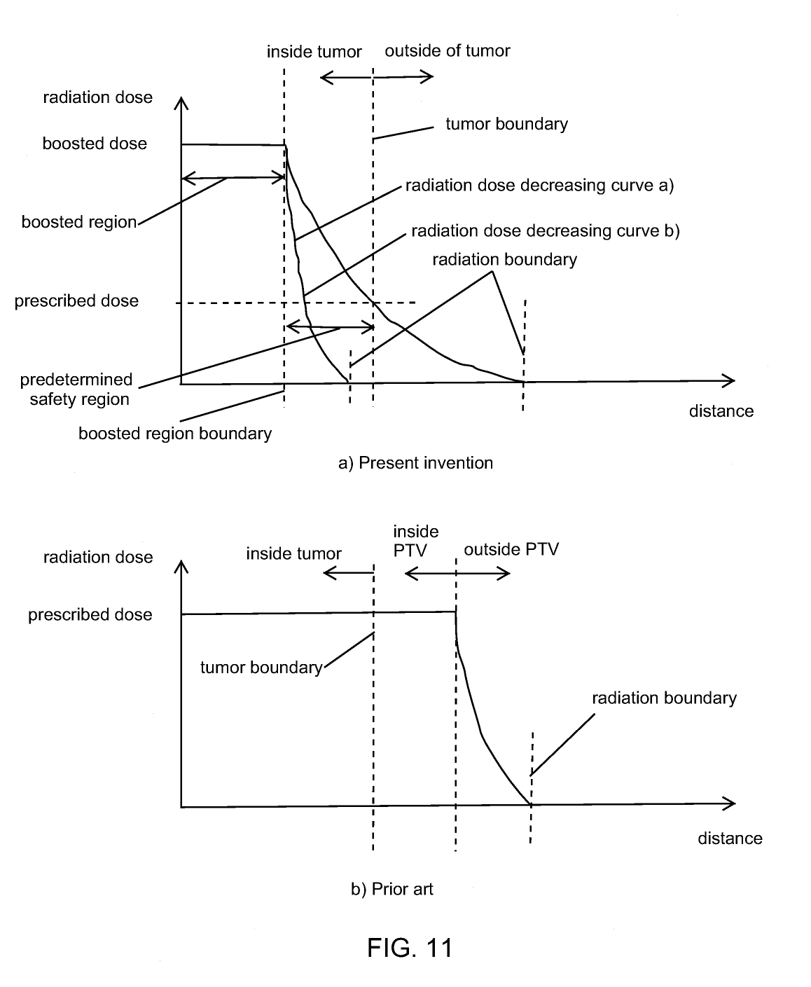

In some embodiment, the method further comprising steps of: a) designating a predetermined prescribed radiation dose of treatment for the boundary of the tumor, wherein the predetermined prescribed radiation dose is smaller than the boosted radiation dose; b) applying radiation treatment for the region safety region (125) with a first dose decreasing rate such that the radiation dose on the tumor boundary is the a predetermined prescribed radiation dose; c) applying radiation treatment beyond the tumor boundary and within the radiation boundary (140) with a second dose decreasing rate; wherein the radiation treatment for the region safety region (125) and beyond the tumor boundary are applied together with the radiation treatment for the boosted region (120). The radiation dose decreasing curve can be viewed in FIG. 11, radiation dose decreasing curve a).

In some embodiment, the radiation dose decreases rapidly beyond boost region boundary and reduces to zero within the safety region (125). The radiation dose decreasing curve can be viewed in FIG. 11, radiation dose decreasing curve b).

In some embodiments, the tumor is radiation sensitive tumors, such as Squamous Carcinoma, Adeno Carcinoma, small cell Carcinoma, Lymphoma Carcinoma or Transitional cell Carcinoma. In some embodiments, the featured image-guided radiotherapy method for tumor treatment is applicable to treatment of neck node less than 3 centimeter (cm) in diameter. In some embodiments, the featured image-guided radiotherapy method for tumor treatment is applicable to treatment of neck nodes with diameter between 3 cm and 6 cm. In some embodiments, the featured image-guided radiotherapy method for tumor treatment is applicable for effective treatment of neck nodes larger than 6 centimeter (cm) in diameter. In some embodiments, the featured image-guided radiotherapy method for tumor treatment is applicable to treatment of locally advanced head and neck cancer that have not spread to distant organs. In some embodiments, the featured image-guided radiotherapy method for tumor treatment is applicable to treatment of locally advanced tumor invading the adjacent organs, such as the spinal cord, blood vessels, brachial plexus or other nerve roots, optic chiasm, optic nerves, pituitary, eyes, small and large bowels, trachea, major airways, kidneys, liver, bladder, and genital organs. In some embodiments, the featured image-guided radiotherapy method for tumor treatment is applicable for effective treatment of a tumor larger than 6 centimeter (cm) in diameter.

As an illustration, a locally advanced head and neck cancer may erode into the spinal canal and threaten to produce paralysis through compression of the spinal cord. The current technique allows for shrinkage of the tumor away from the spinal cord because of the high dose gradient inside the tumor while the spinal cord receives a radiation dose that does not exceed the threshold for damage to the spinal cord. A similar technique can be used to spare the eyes and optic nerves from excessive radiation that can produce blindness when the tumor invades into the orbits. Some tumors such as nasopharyngeal cancers have the propensity to invade the brain through the base of skull and compress the cranial nerves and brain producing paralysis of the face, blindness, deafness, and stroke. The current radiotherapy technique allows for decompression of the nerves when the tumor shrink and may potentially save the patient life and/or quality of life because these tumors are inoperable and requires high radiation dose for cure. This new radiotherapy technique can be applied to any tumor sites in the body because it kills the tumor from the inside and spare the adjacent radiosensitive organs.

In some embodiments, the featured image-guided radiotherapy method for tumor treatment is applicable to treatment of locally advanced head and neck cancer with serious toxicity during radiation.

Radiation therapy dose is measured in Gray or centigray (cGy) (1 Gray=100 cGy). Radiation dose delivered a day is called fraction and conventionally limited to 180-200 cGy a day. In patients with known radio-resistant tumor such as melanoma or renal cancer, radiation dose is increased to 250 cGy or more because of the tumor ability to repair radiation damage. Most tumors often require a total dose of 7000 cGy delivered over six to seven weeks of treatment for possible local control.

In some embodiments, the total energy delivered during one treatment cycle, such as six to seven weeks, with the featured tumor treatment of internal boost is equal to the total energy regulated or specified by the Radiation Therapy Oncology Group (RTOG) with one radiation dose level applied to the planning treatment volume (PTV). In some embodiments, the total energy delivered during one treatment cycle, such as six to seven weeks, with the featured tumor treatment of internal boost is larger than the total energy regulated or specified by the Radiation Therapy Oncology Group (RTOG) with one radiation dose level applied to the planning treatment volume (PTV). With the featured tumor treatment of internal boost, the radiation energy is tuned to focus within internal part of the tutor, rather than distributed uniformly around planning treatment volume, which includes the tutor volume itself and beyond. The total energy is referred as the total radiation energy absorbed for the entire volume where radiation treatment is received.

In some embodiments, the total energy delivered during one treatment cycle, such as six to seven weeks, with the featured tumor treatment of internal boost is equal to the total energy with the traditional radiation treatment method using one radiation dose level applied to the planning treatment volume (PTV). In some embodiments, the total energy delivered during one treatment cycle, such as six to seven weeks, with the featured tumor treatment of internal boost is larger than the total energy with the traditional radiation treatment method using one radiation dose level applied to the planning treatment volume (PTV). With the featured tumor treatment of internal boost, the radiation energy is tuned to focus within internal part of the tutor, rather than distributed uniformly around planning treatment volume, which includes the tutor volume itself and beyond. The total energy is referred as the total radiation energy absorbed for the entire volume where radiation treatment is received.

The boosted region (120) is more than 20% of the volume within the tumor boundary (115). In some embodiments, the boosted region (120) is more than 30% of the volume within the tumor boundary (115). In some embodiments, the boosted region (120) is more than 40% of the volume within the tumor boundary (115). In some embodiments, the boosted region (120) is more than 50% of the volume within the tumor boundary (115). In some embodiments, the boosted region (120) is more than 60% of the volume within the tumor boundary (115). In some embodiments, the boosted region (120) is at least 70% of the volume within the tumor boundary (115). In some embodiments, the boosted region (120) is between 70% and 90% of the volume within the tumor boundary (115). In some embodiments, the boosted region (120) is between 70% and 80% within the tumor boundary (115). In some embodiments, the boosted region (120) is between 80% and 90% within the tumor boundary (115).

In some embodiments, the prescribed radiation dose is between 180 and 200 centiGray (cGy), while the boosted radiation dose is about 250 centiGray (cGy) or above. In some embodiments, the average of the second dose decreasing rate is about 10% per millimeter.

In some embodiments, the average radiation dose in the safety region (125) is 5% less than the radiation dose in the boosted region (120). In some embodiments, the average radiation dose in the safety region (125) is 10% less than the radiation dose in the boosted region (120). In some embodiments, the average radiation dose in the safety region (125) is 15% less than the radiation dose in the boosted region (120). In some embodiments, the average radiation dose in the safety region (125) is 20% less than the radiation dose in the boosted region (120). In some embodiments, the average radiation dose in the safety region (125) is 25% less than the radiation dose in the boosted region (120). In some embodiments, the average radiation dose in the safety region (125) is 30% less than the radiation dose in the boosted region (120). In some embodiments, the average radiation dose in the safety region (125) is more than 30% less than the radiation dose in the boosted region (120).

In some embodiments, the predetermined distance between the boosted region boundary and tumor boundary is uniform. In some embodiments, the predetermined distance between the boosted region boundary and tumor boundary is non-uniform. The predetermined minimum distance (126) between the boosted region boundary and tumor boundary is dependent on the boosted radiation dose and the prescribed radiation dose. In some embodiments, the predetermined minimum distance (126) is about 1 centimeter (cm). In some embodiments, the predetermined minimum distance (126) is between 0.05 and 0.1 cm. In some embodiments, the predetermined minimum distance (126) is between 0.1 and 0.25 cm. In some embodiments, the predetermined minimum distance (126) is between 0.25 and 0.5 cm. In some embodiments, the predetermined minimum distance (126) is between 0.5 and 0.75 cm. In some embodiments, the predetermined minimum distance (126) is between 0.75 and 1 cm. In some embodiments, the predetermined minimum distance (126) is between 1 and 2 cm. In some embodiments, the predetermined minimum distance (126) is between 2 and 5 cm.

In some embodiments, the predetermined distance between the boosted region boundary and tumor boundary is uniform. In some embodiments, the predetermined distance between the boosted region boundary and tumor boundary is non-uniform. The predetermined minimum distance (126) between the boosted region boundary and tumor boundary is dependent on the boosted radiation dose and the prescribed radiation dose. In some embodiments, the predetermined minimum distance (126) is about 1 centimeter (cm). In some embodiments, the predetermined minimum distance (126) is between 0.1 and 0.5 cm. In some embodiments, the predetermined minimum distance (126) is between 0.5 and 1 cm. In some embodiments, the predetermined minimum distance (126) is between 1 and 2 cm. In some embodiments, the predetermined minimum distance (126) is between 2 and 5 cm.

In some embodiments, the method further includes identifying a radiation sensitive normal organ (130) near the tumor boundary (115) and further increasing a distance (128) within the tumor (110) near the organ (130) to avoid excessive radiation influence on the radiation sensitive normal organs (130). In some embodiments, the predetermined minimum distance is dependent on importance level and radiation sensitivity level of the normal organ (130).

Similarly, in the case of radiation resistant tumor treatment, the predetermined minimum distance (126) in the between the boosted region boundary and tumor boundary is dependent on the boosted radiation dose and the prescribed radiation dose. In some embodiments, the boosted radiation dose is between 300-450 centiGray/day or above and the second dose decreasing rate is 10% per millimeter.

Similarly, in the case of radiation resistant tumor treatment, the method further includes identifying a radiation sensitive normal organ (130) near the tumor boundary (115) and further increasing the distance (126) within the tumor (110) near the organ (130) to avoid excessive radiation influence on the normal organs (130).

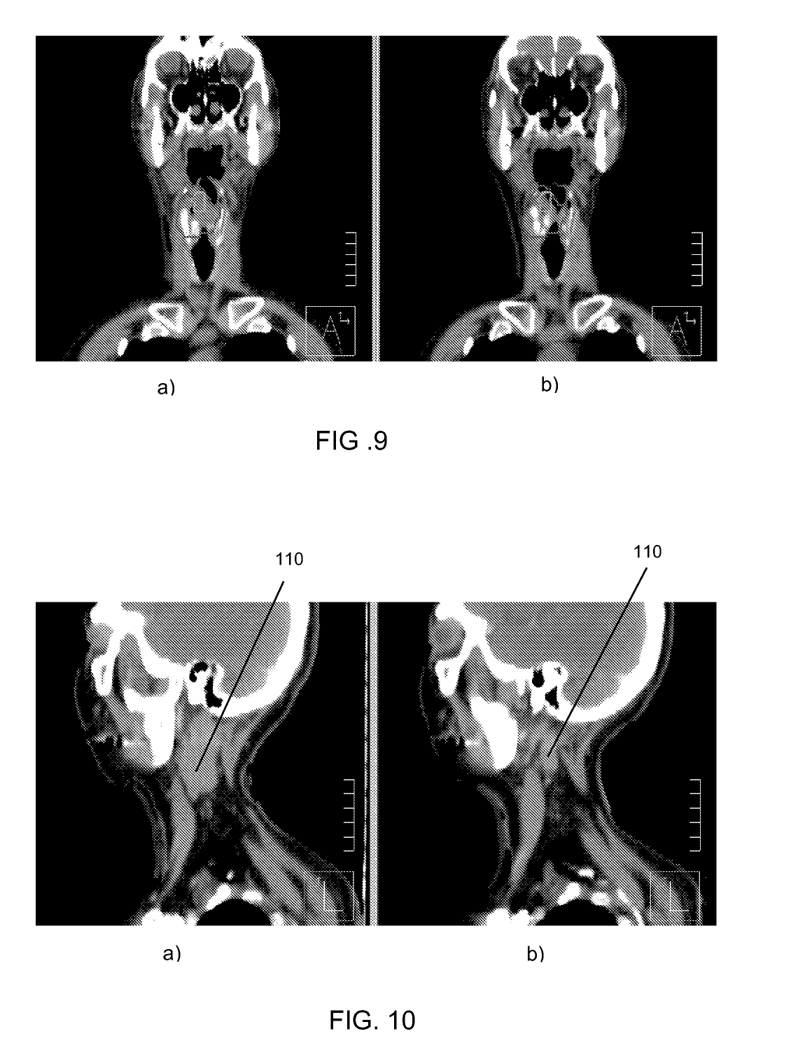

The experimental comparison of treatment using the present method before and after treatment is shown in FIGS. 9 and 10. FIG. 9a) and FIG. 10a) shows the image before treatment in front and side view respectively. FIG. 9b) and FIG. 10b) shows the image after treatment in front and side view respectively. The redline represents the tumor treated to 200 centigray (cGy) a day. The pink line represents the internal boost treated 220 cGy a day (110%). At least 70% of the tumor received 110%. The tumor shrinks during radiation and now there is air (black) inside the tumor because it melts like snow from the high radiation dose.

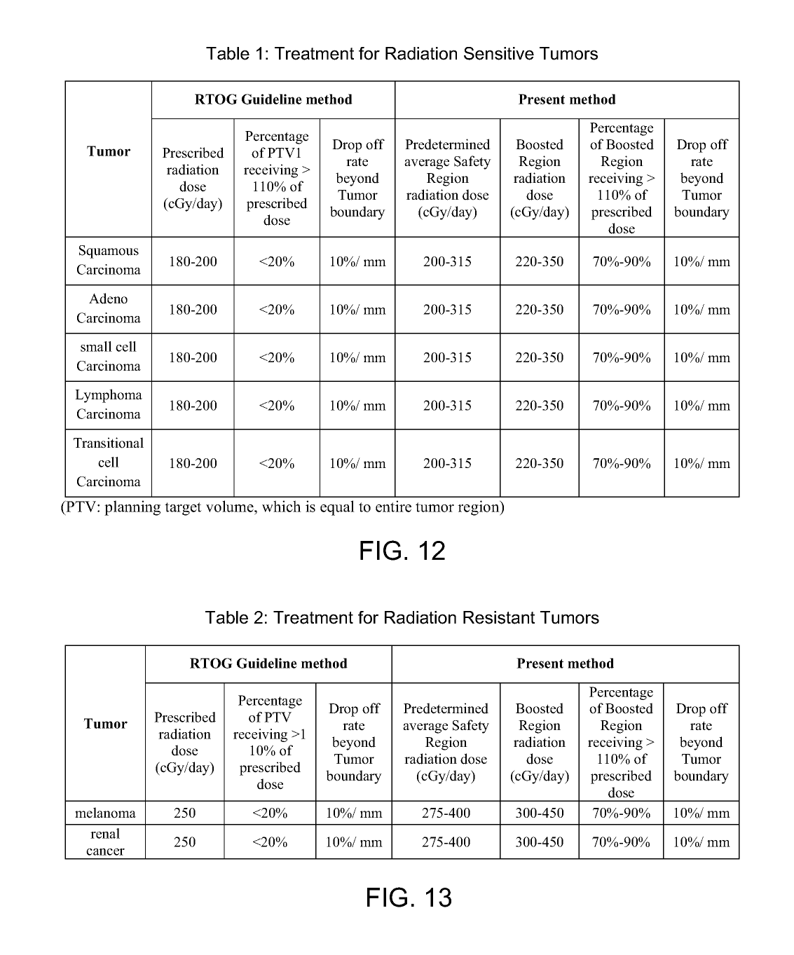

Table 1 in FIG. 12 and Table 2 in FIG. 13 show some typical radiation dose for typical RTOG Guideline method and the present method.

The radiation for use in accordance with the present invention may be delivered using any appropriate beam radiation techniques, for example, Intensity Modulated Radiotherapy (IMRT) and Image-guided radiotherapy (IGRT). With IGRT the clinician can see the pictures of the tumor daily. With IMRT, the clinician cannot see the pictures. Systems which can be used in accordance with the present invention include ones made by Tomotherapy, Varian, Siemens, Elekta, Toshiba and the like. Varian has Varian True Beam, Rapid Arc, Varian EX or IX.

### CLINICAL EXAMPLE 1

In this example, a 75-year old white male patient with a stage T4N2M0 laryngeal cancer (locally advanced tumor that spread to the cervical lymph nodes producing enlargement of the lymph node between 3 to 6 cm and without distant metastases) had been treated with a prescribed tumor dose of 7000 cGy at 200 cGy per day and boosted radiation level of 7700 cGy at 220 cGy/day for 35 days. The original tumor was obstructing the airway and threatened to asphyxiate the patient. After 20 days of treatment (or 4000 cGy prescribed tumor dose and 4400 cGy boosted radiation dose for boosted region), the tumor shrunk to 20% of its initial size allowing the patient to breathe. The treatment has no complication observed. Boosted region is about 80% of the tumor volume. The treatment is repeated daily. At each new treatment, a new scanning for tumor location and size have been done before applying of radiation treatment to ensure the accuracy of the dose delivered. The cancer disappeared after treatment both on clinical exams and diagnostic X-rays. The patient has a normal voice following treatment and remains free of cancer 16 months following treatment.

### CLINICAL EXAMPLE 2

In this example, a 71-year-old patient with a stage T4N0M0 (locally advanced tumor that did not spread to the cervical lymph nodes and distant organs) oropharyngeal cancer had been treated with a tumor dose of 7000 cGy (200 cGy a day) and boosted radiation level of 7700 cGy at 220 cGy/day for 35 days. The tumor extended upward from the soft palate to the nasopharynx, anteriorly to the hard palate and oral cavity, and downward to the base of tongue preventing patient from swallowing food. After 15 days of treatment (or 3000 cGy prescribed tumor dose treatment and 3300 cGy boosted radiation dose for boosted region), the tumor had reduced to 90% of its initial size allowing the patient to swallow again. The treatment has no complication observed. Boosted region is about 85% of the tumor volume. The treatment is repeated daily. At each new treatment, a new scanning for tumor location and size have been done before applying of radiation treatment to verify treatment accuracy. The cancer completely disappeared at the end of radiation treatment. The patient is currently disease free 21 months following treatment and is able to eat and drink normally without any complications.

### CLINICAL EXAMPLE 3

In this example, a 56-year-old white male with a stage T4N3M0 oropharyngeal cancer (locally advanced tumor that invaded the cervical lymph nodes producing enlargement of the lymph nodes more than 6 cm and without distant metastases) had been treated with a dose of 7000 cGy (200 cGy/day) to the tumor and bilateral lymph nodes and boosted radiation level of 7700 cGy (220 cGy/day for 35 days. Original neck nodes measured 8 cm in diameter. After 20 days of treatment (or 4000 cGy prescribed tumor radiation dose and 4400 cGy boosted radiation dose for boosted region), the neck nodes had reduced to a diameter about 3 cm. The treatment has no complication observed. Boosted region is about 70% of the tumor volume and neck nodes. The treatment is repeated each day. At each new treatment, a new scanning for tumor location and size have been done before applying of radiation treatment to verify treatment accuracy. The tumor and neck nodes completely disappeared following treatment and the patient is cancer-free five months after treatment without any complications.

As used herein, the term “about” refers to plus or minus 10% of the referenced number. For example, an embodiment wherein a radiation dose is about 250 centiGray (cGy) includes radiation dose between 225 and 275 centiGray (cGy).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

The reference numbers recited in the below claims are solely for ease of examination of this patent application, and are exemplary, and are not intended in any way to limit the scope of the claims to the particular features having the corresponding reference numbers in the drawings.

What is claimed is:

1. An image-guided radiotherapy method for treatment of a tumor, wherein the method comprising:
(a) obtaining a three-dimensional visualized tumor image (100) to identify a tumor (110);
(b) identifying a tumor boundary (115) of the tumor (110);
(c) designating and applying a boosted radiation dose of treatment for a boost region (120) within the tumor boundary, wherein a predetermined safety region (125) is between the boosted region and the tumor boundary (115) with a predetermined minimum distance (126) between the boosted region boundary and tumor boundary, wherein the boosted region (120) is more than 20% of the volume within the tumor boundary (115); and
(d) repeating steps (a) through (c) for new treatment each time.

2. The method of claim 1, wherein the method further comprising:
(a) designating a predetermined prescribed radiation dose of treatment for the boundary of the tumor, wherein the predetermined prescribed radiation dose is smaller than the boosted radiation dose;
(b) applying radiation treatment for the region safety region (125) with a first dose decreasing rate such that the radiation dose on the tumor boundary is the a predetermined prescribed radiation dose;
(c) applying radiation treatment beyond the tumor boundary and within the radiation boundary (140) with a second dose decreasing rate; and
wherein the radiation treatment for the region safety region (125) and beyond the tumor boundary are applied together with the radiation treatment for the boosted region (120).

3. The method of claim 1, wherein the tumor is Squamous Carcinoma, Adeno Carcinoma, small cell Carcinoma, Lymphoma Carcinoma or Transitional cell Carcinoma.

4. The method of claim 1, wherein the boosted region (120) is at least 70% of the volume of the volume within tumor boundary (115).

5. The method of claim 2, wherein the predetermined minimum distance between the boosted region boundary and tumor boundary is dependent on the boosted radiation dose and the prescribed radiation dose.

6. The method of claim 1, wherein the boosted radiation dose is about 250 centiGray (cGy) or above.

7. The method of claim 1, wherein the prescribed radiation dose is between 180 and 200 centiGray (cGy).

8. The method of claim 1, wherein the average of the second dose decreasing rate is about 10% per millimeter.

9. The method of claim 1, wherein the method further includes identifying a radiation sensitive normal organ (130) near the tumor boundary (115) and further increasing a distance (128) within the tumor (110) near the organ (130) to avoid excessive radiation influence on the radiation sensitive normal organs (130).

10. An image-guided radiotherapy method for treatment of a radiation resistant tumor, wherein the method comprising:
(a) obtaining a three-dimensional visualized tumor image (100);
(b) identifying the boundary (115) of the tumor (110);
(c) designating and applying a boosted radiation dose of treatment for a boost region (120) within the tumor boundary, wherein a predetermined safety region (125) is between the boosted region and the tumor boundary (115) with a predetermined minimum distance between the boosted region boundary and tumor boundary, wherein the boosted region (120) is more than 20% of the volume within the tumor boundary (115);
(d) designating a predetermined prescribed radiation dose of treatment for the boundary of the tumor, wherein the predetermined prescribed radiation dose is smaller than the boosted radiation dose;
(e) applying radiation treatment for the region safety region (125) with a first dose decreasing rate such that the radiation dose on the tumor boundary is the a predetermined prescribed radiation dose;
(f) applying radiation treatment beyond the tumor boundary and within the radiation boundary (140) with a second dose decreasing rate; and
(g) repeat steps (a) thru (f) for new treatment each time.

11. The method of claim 10, wherein the predetermined minimum distance (126) between the boosted region boundary and tumor boundary is dependent on the boosted radiation dose and the prescribed radiation dose.

12. The method of claim 10, wherein the radiation resistant tumor is melanoma or renal cancer.

13. The method of claim 10, wherein the boosted radiation dose is between 300 centiGray/day and 450 centiGray/day.

14. The method of claim 10, wherein the average of the second dose decreasing rate is about 10% per millimeter.

15. The method of claim 10, wherein the method further includes identifying a radiation sensitive normal organ (130) near the tumor boundary (115) and further increasing the distance (126) within the tumor (110) near the organ (130) to avoid excessive radiation influence on the normal organs (130).
